(19) United States
(12) Patent Application Publication
(10) Pub. No.: US 2026/0199046 A1
(43) Pub. Date: Jul. 16, 2026

(54) SURGICAL SAFETY APPARATUS

(71) Applicant: Eureka Inventions Limited, Newcastle, Staffordshire (GB)

(72) Inventors: Aditi DESAI, Newcastle, Staffordshire (GB); Kiran DESAI, Newcastle, Staffordshire (GB)

(21) Appl. No.: 19/140,970

(22) PCT Filed: Dec. 21, 2023
(86) PCT No.: PCT/GB2023/053356

§ 371 Date: Jun. 18, 2025

(30) Foreign Application Priority Data
Dec. 23, 2022 (GB) 2219738.8

(51) Int. Cl. A61B 50/20; A61B 90/00

(52) U.S. Cl. CPC A61B 50/20; A61B 90/08; A61B 2090/0805

(57) ABSTRACT

An apparatus is provided for use in a surgical procedure. The apparatus includes a dock for releasably securing a surgical device by a releasable tether of the device. The dock includes one or more ports. Each port is adapted such that when a tether is secured, a visible indication of attachment or detachment is provided by the port or the tether.

The invention relates to surgical safety apparatus, and more particularly apparatus and a method for use in the prevention of retention of foreign objects in patients, post-surgical procedure.

According to the publication “Provisional Publication of Never Events reported as Occurring between 1 April and 31 Oct. 2022” published on the 8 Dec. 2022 by NHS England, out of 243 Never Events, 58 events fell under the category “Retained Foreign Object Post Procedure”, and of those the two events with the highest incidence involved retained surgical swabs (10) and retained vaginal swabs (18).

Despite the establishment of a formal NHS Patient Safety Strategy in 2019, overall levels of the occurrence of Never Events have not reduced significantly and retained foreign objects post-procedure continues to be one of the most frequently occurring.

In their patent application WO2020/084313, the inventors have described concepts and apparatus aimed at addressing such issues, and the solutions so presented have contributed to their alleviation. Substantial problems still remain, however, and a recurrent theme is the role of human factors in such events. While strong systemic barriers are recognized in the NHS Patient Safety Strategy as being of key importance in preventing Never Events it is also recognized that for some categories of event, design solutions that strengthen barriers are also needed in order to further assist in mitigating the effects of these so-called human factors. In particular, optimizing human-centred design of medical equipment, medical devices, EPMA, electronic patient records, healthcare apps, etc. to improve patient safety has been recognized as key because it addresses one simple but key human factor issue, that of non-usage. The most technically innovative and successful design of device will be ineffective if it is never used, and in general the more technically complicated and involved a device is, the less likely it is to be used, particularly in a busy, high stress environment like an operating theatre.

In the publication, “Reflecting on why Surgical Swabs are Being Left Behind and Exploring how this Could be Prevented”, (BJM, volume 29, Issue 6, 2 Jun. 2021) Spranzi suggests the introduction of an anchoring point linking together the five swabs normally present in a swabs pack. Each swab includes a web of a length suitable to allow for use in a patient. The webs are linked by a sewn portion at their proximal ends which remains outside of the patient. The feature of permanent linkage is a benefit in that it makes it less likely that swabs will be left behind, but in some instances it can also be a drawback because the connected webs protruding from the patient may impede the work of the clinicians.

EP 1933795, EP 2399559, U.S. Pat. Nos. 7,795,491 and 7,703,674 relate to a system comprising barcodes that may be attached to swabs.

It is an object of the present invention to seek to provide improved surgical safety apparatus that addresses the issue of non-use through complexity, whilst still achieving increased patient safety.

According to a first aspect of the invention, there is provided apparatus for use in a surgical procedure, the apparatus comprising a dock for releasably securing a surgical device by means of a releasable tether of the device, the dock including one or more port, the or each port being adapted such that when a tether is secured, a visible indication of attachment or detachment is provided by the port or the tether. Thus, the invention mitigates against the never event of “retained foreign object post-procedure” whilst also being simple to operate, and allows for separation and manipulation of surgical devices as necessary during a procedure by clinicians.

It is preferred that the apparatus includes one or more clip, the or each clip being adapted for attachment to the proximal, in use, end of a tether of each surgical device, the or each clip being adapted to releasably connect the tether to the dock.

In one embodiment, the dock may consist solely of a plurality of clips, each clip being adapted to connect with each other clip such that when all clips are connected, a complete dock is provided, the visible indication of the absence of a clip being provided by a gap between clips. As will be appreciated, in this embodiment the clips and ports are one and the same and as a result the apparatus is less complex and therefore less expensive to manufacture.

It is preferred that the or each clip is removably attachable to a tether, allowing for re-use of clips.

In order for the apparatus to be ergonomically optimised for use, it is preferred that it is adapted such that in use, connected tethers will be disposed so that they are substantially parallel with one another. This can be achieved, for example by an arrangement wherein ports are arranged along a single side or face of the apparatus. It is also preferred that where the apparatus is used with substantially planar tethers, such as for example are present on surgical swabs, each tether is connected to the apparatus in the same plane.

In another embodiment, the dock may comprise a single body part, the part including a plurality of integrally moulded ports. The or each port may be configured such that a clip is receivable therein and retained therein through the resilience of moulded material of the ports, or of the clips.

In one preferred arrangement, each port may include a user visible aperture disposed to receive a correspondingly shaped formation of a clip, the aperture being filled by a part of the clip when the clip is correctly inserted in the port. It is further preferred that the parts are adapted such that release of a clip is effected by a user action applied to the part of the clip in the aperture. This arrangement provides a visible indication that a clip is correctly positioned in its port, so that unintentional release and loss of the surgical device is avoided. In one particularly preferred embodiment, the ports may comprise resilient arms describing a part circle and the corresponding clips may comprise cylinders configured to fit within the arms. Each cylinder may include a circumferential groove disposed to receive the arms therein. Alternatively, each port may comprise a clip receiving aperture and a clip release aperture, and each corresponding clip may comprise a release part adapted to engage with the release aperture and a tether securing location.

In another preferred arrangement, each port may include a keeper adapted to be moved from a first unlocked condition to a second, locked condition, the arrangement being such that the keeper can only assume the locked condition when a clip is in situ in the port. It is preferred that the keeper and/or port is configured such that the position of the keeper, i.e. locked or unlocked, is visible to a user. In one arrangement, the or each keeper may take the form of a door, openable about a hinge, the or each door including an aperture through which an associated clip may be viewed.

According to another embodiment, the dock may comprise a tray. In one embodiment, the tray may comprise a lid. Preferably, the tray comprises a plastics material. Typically, the tray may comprise one or more recess, wherein the one or more recess is adapted to hold the one or more clip. Preferably, the one or more clip may be secured to the dock. Advantageously, the one or more clip may be attached to a swab during a surgical procedure, allowing the user to visualize how many swabs have been accounted for to ensure that no swabs are inadvertently left in an incorrect position following the procedure.

In another embodiment of the invention, the dock may comprise a card for storing the one or more clip before and/or after a surgical procedure. Advantageously, the card provides a storage means for the one or more clips before a surgical procedure and provides a means of visualizing the clips following the procedure to ensure that no clips which have been attached to a swab are unaccounted for following the procedure.

In another embodiment, the dock may comprise a clip comprising a plurality of recesses, wherein each recess is adapted to store one or more swabs. In this embodiment, the one or more swabs may be placed within the one or more recesses and, once in place, the clip may be closed to retain the one or more swabs.

Preferably, the clip and/or dock comprises a mechanism such that it will not close if one or more swabs is not engaged within a corresponding recess.

Preferably, the clip comprises a metallic material which advantageously may be sterilized. In one embodiment, the clip may be autoclaved.

In one embodiment, the clip may be reusable. Typically, the clip may have smooth outer surfaces. Advantageously, the provision of smooth outer surface on the clip may assist with the sterilization of the clip. Typically, it may be easier to clean the clip when it is in an open configuration. In one embodiment the clip may be cleaned with water after use and then may be sterilized.

Preferably, the one or more recess is lined with a material which assists with gripping and holding the swab in place within the recess. In one embodiment, the one or more recess is lined with silicone.

Preferably, the clip comprises an upper arm and a lower arm, wherein the upper and lower arm are joined by a hinged region. Preferably, the end of the upper and lower arm furthest from the hinged region comprises a clipping portion which preferably comprises a latch provided on the upper arm and a corresponding projection or recess provided on the lower arm wherein the projection or recess on the lower arm engages with the latch and thus holds the clip in a closed position.

In another embodiment, the clip comprises a button which is adapted to release the swabs from the one or more recess, for example, when the swabs are to be disposed. In one embodiment, the user may press the button to open the clip to allow the release of the swabs. Advantageously, the button may be adapted to release the swabs from the clip so that the swabs may be sterilized.

According to a second aspect of the invention there is provided a kit for use in surgery, comprising apparatus as described above and a plurality of surgical devices, each surgical device including a tether receivable in a clip.

According to a third aspect of the invention there is provided a surgical method, comprising the step of tethering surgical devices when in use in a procedure, to apparatus as described herein above.

According to a fourth aspect, there is provided a system comprising a dock and card combination to monitor the removal of swabs following a surgical procedure.

Preferably, the system comprises one or more clips that may be removably attached to the card. Advantageously, the card provides a storage means for the one or more clips before a surgical procedure and provides a means of visualizing the clips following the procedure to ensure that no clips which have been attached to a swab are unaccounted for following the procedure.

According to a fifth aspect there is provided an image recognition system for detecting and/or counting the number of swabs within a dock in accordance with the first aspect, the system comprising camera means, computer processing, image recognition means with or without AI software.

Preferably, the system comprises a clip.

In one embodiment, the system may be connected to an electronic records system in respect of a patient.

Preferably, the system is in communication with an app. Preferably, the system is programmed so as to know the correct number of swabs that should be present. It is preferred that the system does not allow data to be uploaded to the electronic records system until the correct number of swabs are present. Advantageously, the system allows for the independent verification of the number of swabs that are present.

Preferably, the clip and/or dock comprise a visual identifier to enable them to be distinguished from a swab by the camera. Advantageously, the provision of a visual identifier on the clip and/or dock assists the user in identifying a swab after use (after use of a swab it can be difficult for a medical professional to see the swab since it is typically soiled with blood from a patient). Typically, the clip and/or dock are coloured in a manner such that they are distinguished from the swab and can be seen by the camera. In one embodiment, the clip and/or swab comprise a material having a bright colour that provides a contrast with the background to assist the camera in detecting the clip and/or swab.

In one embodiment, the system comprises a means for identifying if there is blood on the clip and/or swab so that the blood can be removed from the clip and/or swab. Preferably, the system only allows data to be uploaded when the correct number of swabs are present. This advantageously provides an independent means of checking that all of the swabs are present.

In one embodiment, the clip may comprise machine-readable information. In one embodiment the machine-readable information may be a barcode or a QR code which is printed on the clip and/or dock. Advantageously, the system provides a digital counter to determine the number of swabs that are attached to the dock. Preferably, the swabs are provided in a recess of the dock and once the correct number of swabs have been added the one or more clips are closed. Preferably, the closed clip comprises machine-readable information that may be read by the system.

The invention will be further illustrated by way of examples, described with reference to the accompanying drawings, in which:

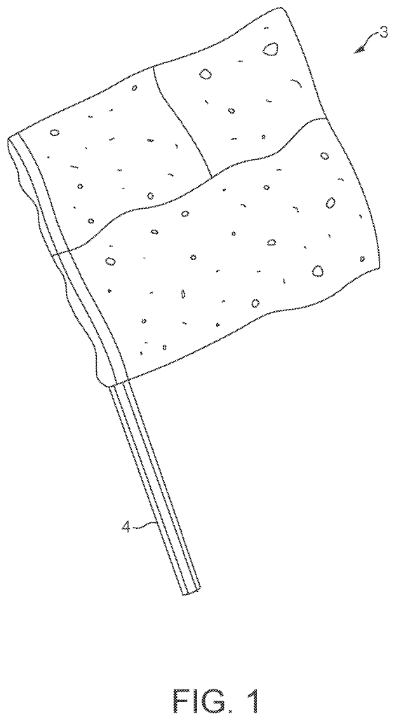

FIG. 1 illustrates an example of a surgical device for use with the apparatus of the invention;

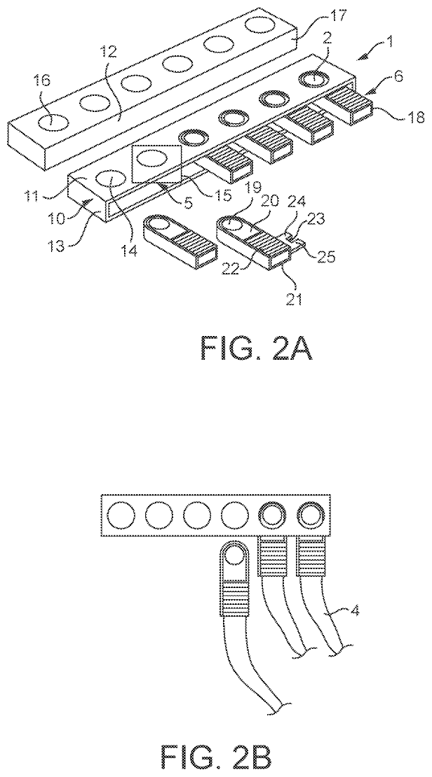

FIGS. 2a and 2b show a perspective view and a top plan view of apparatus according to a first embodiment of the invention;

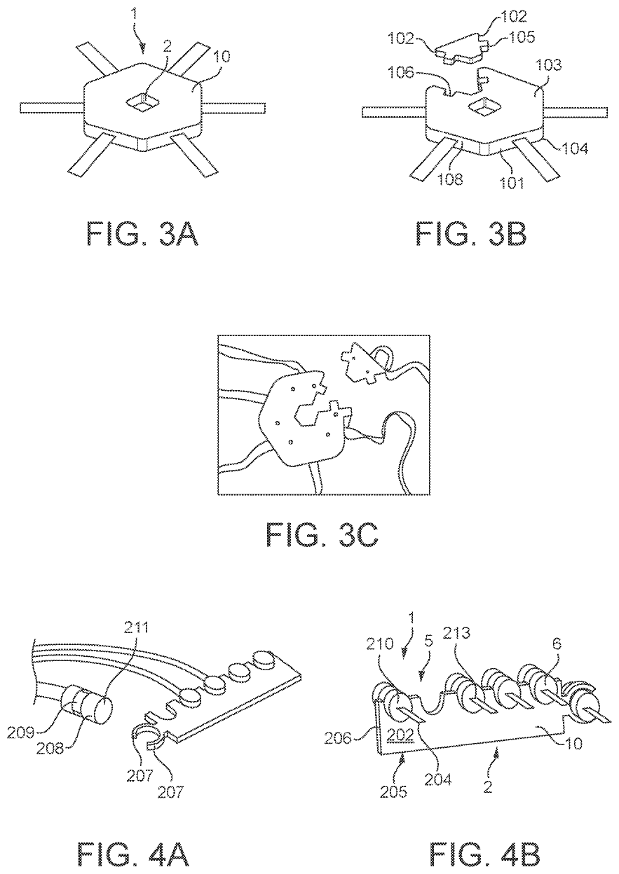

FIGS. 3a to 3c show perspective and top plan views of apparatus according to a second embodiment of the invention;

FIGS. 4a and 4b show perspective views of apparatus according to a third embodiment of the invention;

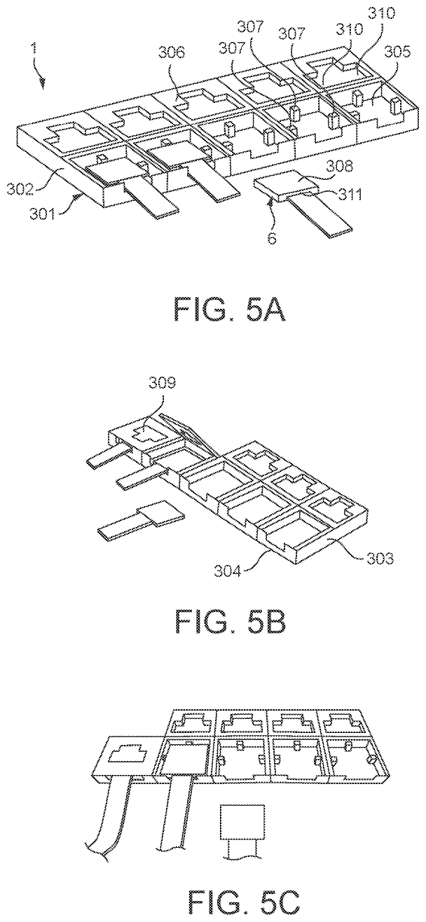

FIGS. 5a to 5c show perspective views of apparatus according to a fourth embodiment of the invention;

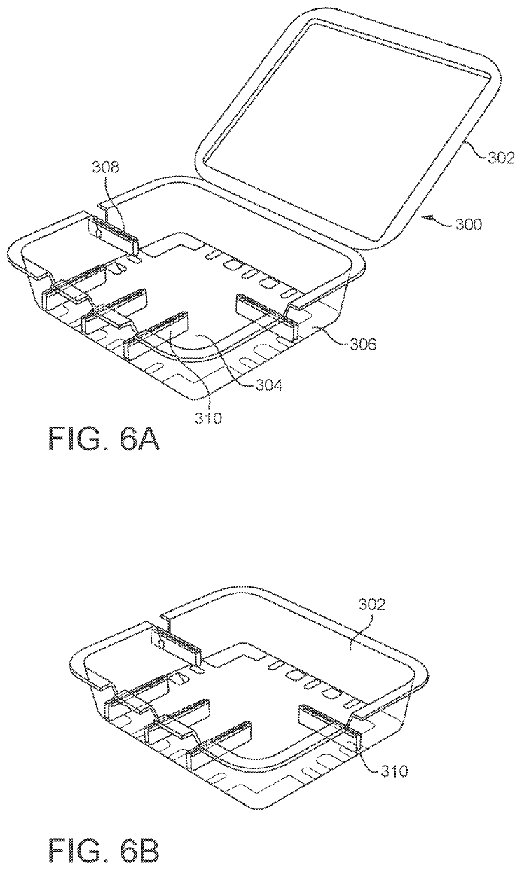

FIG. 6a illustrates a perspective view of apparatus in accordance with a further embodiment of the invention, wherein the apparatus comprises a lid that is shown in an open configuration;

FIG. 6b shows a perspective view of the apparatus of FIG. 6a wherein the lid is shown in a closed configuration;

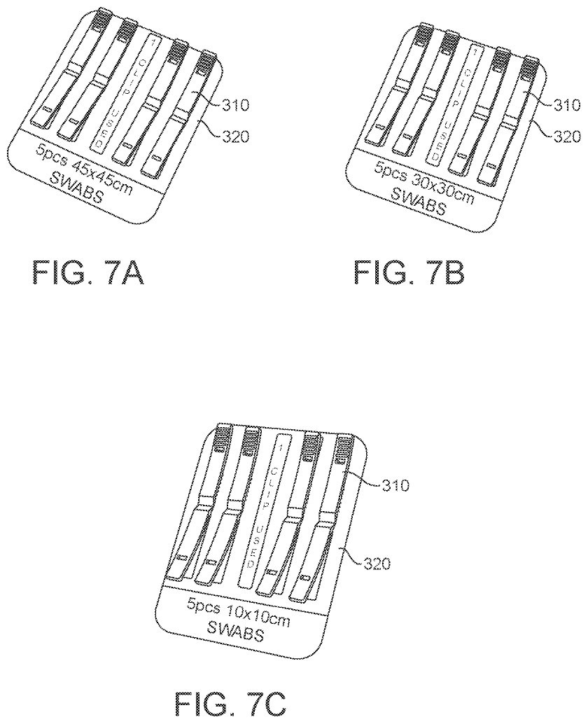

FIGS. 7a, 7b and 7c show a perspective view of apparatus according to different embodiments of the invention;

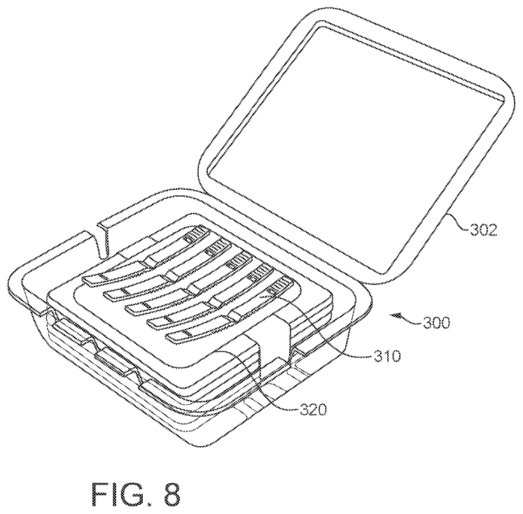

FIG. 8 illustrates a perspective view of apparatus according to another embodiment of the invention;

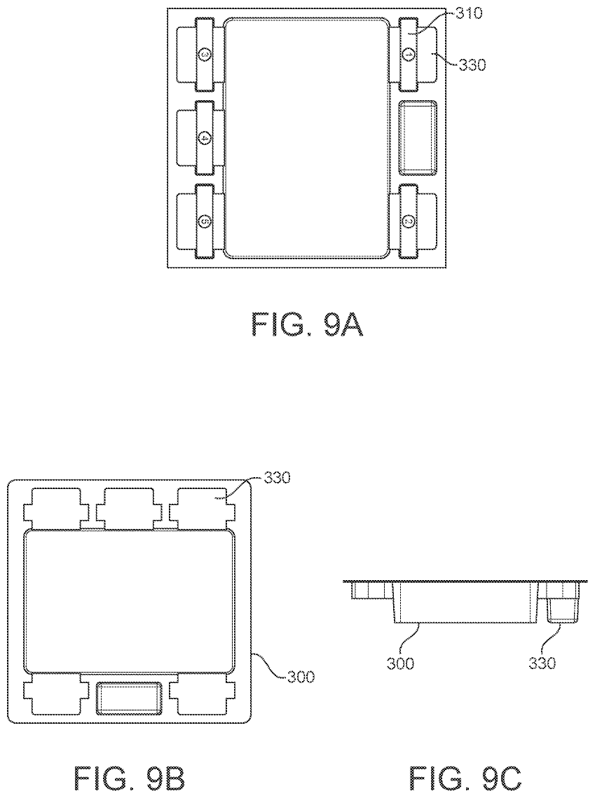

FIG. 9a shows a top view of apparatus according to another embodiment of the invention;

FIG. 9b shows a top plan view of the apparatus of FIG. 9a;

FIG. 9c shows a side view of the apparatus of FIG. 9a;

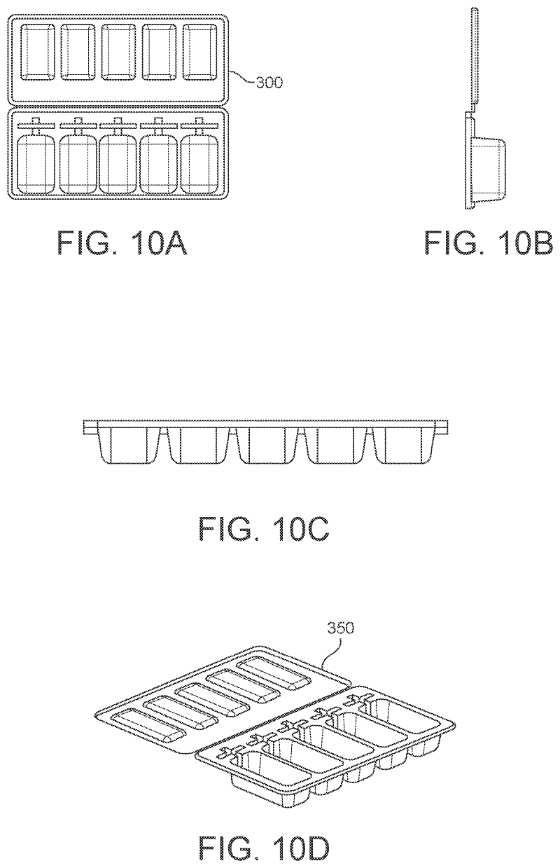

FIG. 10a shows a top plan view of a further embodiment according to the invention;

FIGS. 10b and 10c show side views of the embodiment shown in FIG. 10a;

FIG. 10d shows a perspective view of the embodiment shown in FIG. 10a;

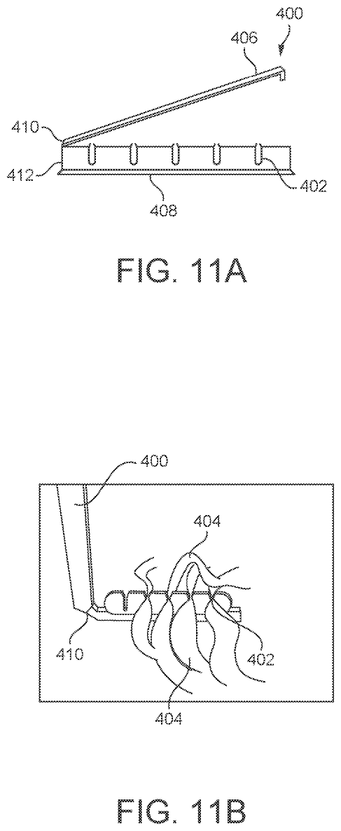

FIG. 11a shows a plan side view of apparatus according to another embodiment of the invention;

FIG. 11b shows a perspective view of apparatus according to the embodiment of FIG. 11a

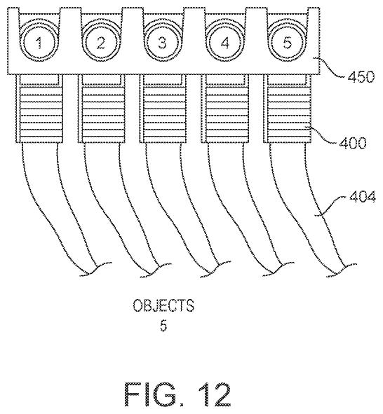

FIG. 12 shows a front view of a system according to an embodiment of the invention;

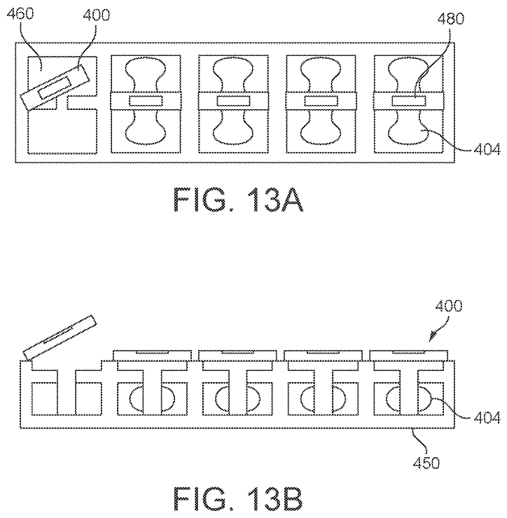

FIG. 13a shows a top view of a system according to another embodiment of the invention;

FIG. 13b shows a side view of the system of FIG. 13a; and

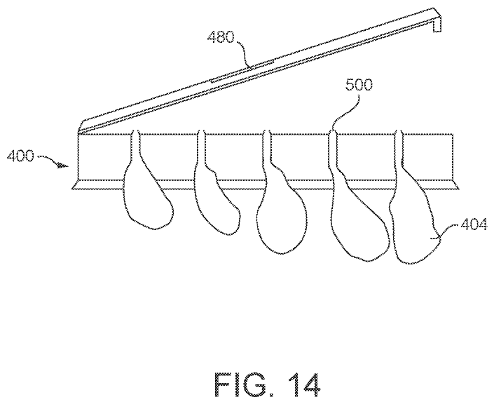

FIG. 14 shows a side view of a system according to another embodiment of the invention.

Referring to the drawings, there is provided apparatus 1 for use in a surgical procedure, the apparatus comprising a dock 2 for releasably securing a surgical device 3 (FIG. 1) by means of a releasable tether 4 of the device, the dock including one or more port 5, the or each port being adapted such that when a tether is secured, a visible indication of attachment or detachment is provided by the port or the tether.

Turning firstly to FIGS. 2a and 2b, a first embodiment of apparatus 1 is illustrated. The apparatus here consists of a generally rectangular square section hollow body 10 which in this embodiment provides the dock 2. The body 10 includes a top surface 11, a bottom surface, a rear wall 12 and two short end walls 13. Instead of a front (in use and as viewed) wall, the body defines five apertures 14 each of which leads into a corresponding square section bore 15 in the hollow interior of the body. The top surface 11 is substantially planar and includes five equally sized circular apertures 16 arranged at regular intervals across its surface and each in register with a bore 15. Each bore 15 and its associated apertures 14, 16 constitutes a port 5. The rear wall 12 is substantially flat, except for the provision of surface connecting parts 17.

In this embodiment, the apparatus 1 includes clips 6, which are provided to create a releasable connection between the dock 2 and tethers 4, as illustrated in FIG. 2b. Each clip consists of a body 18 which is sized and shaped to fit closely within a bore 15. A top, as viewed, surface of each clip includes a circular button 19 adjacent a distal end thereof, which can be depressed into a hollow inner part of the body 18. Button 19 is connected to the body 18 by means of a resiliently depressible flat 20. Adjacent its proximal end each clip is provided with a tether attachment section 21 which here consists of a tether locating surface 22 and hinged flap 23, both of which include protrusions 24 on their inner, in use, surfaces, and locking formations 25 which allow the flap to be releasably locked in the closed position.

As will be appreciated, in FIG. 2a two docks are illustrated, back to back.

In use, surgical devices 3 are firstly attached to clips 6 by means of tethers 4. The end of a tether 4 is placed into the tether attachment section 21 of a clip and hinged flap 23 is closed and locked via locking formations 25. Protrusions 24 assist in preventing the tether from coming loose.

Each clip 6 can then be releasably secured to a dock 2 by inserting it into a bore 15. As the clip is inserted, button 19 is partially depressed by the mouth of bore 15 against the force of flat 20 until the button 19 comes into register with an aperture 14, at which point it is allowed to spring back, thereby holding the clip in place against accidental removal. The corresponding size and shape of the bore and clip and the position of the button 19 ensure that insertion takes place in the correct orientation. When it is desired to remove clip 6, button 19 is depressed allowing the clip 6 to be pulled outwardly from the bore 15.

Referring in particular to FIG. 2a, a certain degree of modularity is achieved by provision of one additional port in addition to the usual five. The additional port 5 can be provided as a snap-off portion of body 10 if desired. In addition, two docks can be combined using surface connecting parts 17.

All parts of the apparatus shown in this embodiment can conveniently be made by injection moulding from a single plastics material, or alternatively, the dock 2 can be moulded from a plastics material of relatively harder Shore D hardness and the clips can be moulded from a plastics material of relatively softer Shore D hardness, in order to achieve correct functioning of the depressible buttons 19, sterilisability and longevity.

Referring now to FIGS. 3a to 3c, a second embodiment of apparatus 1 is illustrated. The apparatus here consists of a hexagonal, planar body 10 which forms the dock 2. The body 10 comprises six clip segments, each segment being generally triangular and including a front face, 101, side faces 102 and bottom and top faces 103, 104. Each segment includes connection formations on its side faces, which can be either male formations 105 or female formations 106. Each segment further includes a tether attachment section 107, including a tether port 108.

As will be immediately apparent, this embodiment differs from the embodiment of FIGS. 2a and 2b in that it does not include separate dock and clip parts. Instead, here the clips 6 together provide the dock 2 when connected together.

In use, surgical devices 3 are firstly attached to clips 6 by means of tethers 4. The end of a tether 4 is placed into the tether attachment section 107 of a clip 6 via tether port 108. When all devices are tethered, the clips can be combined together by means of corresponding male and female formations 105, 106 to form a single dock 2.

The clip segments can be conveniently made by injection moulding a suitable plastics material. As there is no separate dock 2, the apparatus in this embodiment is easier and less costly to manufacture.

Referring now to FIGS. 4a and 4b, a third embodiment of apparatus 1 is illustrated. The apparatus here consists of a generally planar rectangular body 10 which forms the dock 2. The body 10 includes top and bottom surfaces 202, 203, front and rear long edges 204, 205 and short edges 206. Ports 5 are provided along front edge 204 which here take the form of pairs of arms 207, each pair forming a part circle. In this embodiment an additional port 5 is also formed on one of the short edges 206.

In this embodiment, the apparatus includes clips 6, which are provided to create a releasable connection between the dock 2 and tethers 4 as illustrated in FIG. 4a. Each clip consists of a body 208 which here takes the form of a cylinder including an equatorial groove 209 and front and back end faces 210, 211. Each groove 209 is provided in size and shape to correspond with arms 207 of ports 5. Front end face 210 includes a tether attachment section 212 including tether port 213.

In use, surgical devices 3 are firstly attached to clips 6 by means of tethers 4. The end of a tether 4 is placed into the tether attachment section 212 of a clip 6 via tether port 213. Each clip 6 can then be releasably secured to the dock 2 by inserting it through the aperture between the arms 207 of a port 5, which action will momentarily force the arms apart. This is most conveniently done at the equatorial groove 209, such that when the clip 6 is fully received in the port 5, the arms rest within the groove 209.

All parts of the apparatus shown in this embodiment can conveniently be made by injection moulding from a single plastics material, or alternatively, the dock 2 can be moulded from a plastics material of relatively harder Shore D hardness and the clips can be moulded from a plastics material of relatively softer Shore D hardness, in order to achieve correct functioning of the resilient arms 207, sterilisability and longevity.

Referring now to FIGS. 5a to 5c, a fourth embodiment of apparatus 1 is illustrated. The apparatus here consists of five generally rectangular shallow trays 301 which in this embodiment together provide the dock 2. Each tray 301 consists of side walls 302, a rear wall 303 and a front wall 304 which together define a central interior volume 305. Located within the central volume 305 are clip retaining formations 307. The trays are connected in side by side arrangement via their side walls 302. The rear wall 303 of each tray is provided with a keeper in the form of a hinged lid 306 that extends therefrom forwardly and is sized to extend over and thereby cover the central interior volume 305 in a closed position (FIG. 5b). The lid 306 includes an aperture 309 and resilient clip locating formations 310 which extend down, as viewed, into the central volume 305. The hinge of the lid may conveniently be a web of a resilient material which biases the lid to an open position, sometimes known as a “live hinge”. Each tray 301 constitutes a port 5 in this embodiment.

As with the previous embodiments, the apparatus 1 includes clips 6, which are provided to create a releasable connection between the dock 2 and tethers 4 as illustrated in FIG. 5c. Each clip consists of a body 308 which here takes the form of upper and lower generally square body faces connected along an edge and including a tether port 311 along the opposite edge.

In use, surgical devices 3 are firstly attached to clips 6 by means of tethers 4. The end of a tether 4 is placed into the clip 6 between the upper and lower body faces and trapped there between, protruding via tether port 311. Each clip 6 can then be releasably secured to the dock 2 by being inserted into a port 5 when the hinged lid is in the open position. The clip body 308 is placed within the clip retaining formations 307 which serve to correctly position it within the central volume 305 and the hinged lid is moved to the closed position against the action of the bias. When the lid is depressed towards the central volume the clip locating formations 310 of the lid descend into register with the sides of the clip body 308 and are retained by way of an interference fit, thereby keeping the lid closed against the force of the biased hinge.

All parts of the apparatus shown in this embodiment can conveniently be made by injection moulding from a single plastics material, or alternatively, the dock 2 can be moulded from a plastics material of relatively harder Shore D hardness and the clips can be moulded from a plastics material of relatively softer Shore D hardness, in order to achieve correct functioning of the resilient clip locating formations 310, sterilisability and longevity.

With reference to FIGS. 6a and 6b, the apparatus comprises a tray 300 having a lid 302 which may be in the open or closed configuration. In the embodiment shown in FIG. 6a the lid is in an open configuration and in the embodiment shown in FIG. 6b the lid is in a closed configuration. The tray is substantially rectangular in shape and comprises a base 304 having a plurality of walls 306. Preferably, the tray comprises a plastics material. A plurality of recesses 308 are provided in the upper region of the wall of the tray. A clip 310 is preferably inserted into each of the recesses and secured in place. The clips 310 may be attached to a swab during a surgical procedure. Advantageously, the tray allows the user to determine whether any swabs are unaccounted for following the procedure.

FIGS. 7a, 7b and 7c show an embodiment of the invention wherein a card 320 is provided which may be used to store the clips 310 before and/or after a surgical procedure. In one embodiment, the card may comprise paper or card, in another embodiment the card may comprise a plastics material. Preferably, the clips are provided to the user in an embodiment wherein they are attached to the card. As the clips are used during a surgical procedure they are removed from the card 320 and applied to a swab. After the surgical procedure, the clips are then reattached to the card. Advantageously, this arrangement means that the clips can be accounted for after the surgical procedure, ensuring that none of the clips are inadvertently left within the patient. In one embodiment, the clips may comprise a plastics material. In another embodiment, the clips may comprise a metallic material.

FIG. 8 shows an embodiment wherein the tray 300 is provided in combination with the card 320 comprising a plurality of clips 310. Prior to a surgical procedure, the clips and card are removed from the tray. The clips may then be secured within the recesses provided within the upper portion of the tray wall, e.g. as illustrated in FIGS. 6a and 6b.

The tray and/or clips of this embodiment may comprise a plastics material

FIGS. 9a, 9b and 9c show another embodiment of the invention wherein the tray 300 comprises a plurality of wells 330. The wells 330 are provided along the edge of two parallel outer walls of the tray 300. A clip 310 is then placed within each of the wells of the tray. In the embodiment shown in FIG. 9a, the clips are provided such that they lie across the wells of the tray. In use, the user attaches a clip to a swab during a surgical procedure and following the procedure, the clips are placed across the wells of the tray. The embodiment shown in FIG. 9a to 9c provides another arrangement by which the user may visualize the clips and thus swabs that have been used following a surgical procedure.

FIG. 10a to 10d illustrates a further embodiment of the invention wherein the tray 300 comprises a plurality of recesses which may be used to accommodate the clips. The tray may comprise a lid portion 350 to seal the tray, for example, for hygiene purposes.

With reference to FIGS. 11a and 11b, the dock 450 comprises a clip 400 comprising a plurality of recesses 402, wherein each recess is adapted to store one or more swabs 404. FIGS. 11a and 11b show a reusable swab counter. In this embodiment, the one or more swabs are placed within the one or more recesses and, once in place, the clip may be closed to retain the one or more swabs. The clip comprises a mechanism such that it will not close if one of the swabs is not engaged within a corresponding recess. In this embodiment, the one or more recess is lined with a material such as silicone which assists with gripping and holding the swab in place within the recess. As shown in FIGS. 11a and 11b, the clip comprises an upper arm 406 and a lower arm 408, wherein the upper and lower arm are joined by a hinged region 410. Preferably, the end of the upper and lower arm furthest from the hinged region comprises a clipping portion which preferably comprises a latch provided on the upper arm and a corresponding projection or recess provided on the lower arm wherein the projection or recess on the lower arm engages with the latch and thus holds the clip in a closed position. In another embodiment, the clip comprises a button 412 which is adapted to release the swabs from the one or more recess, for example, when the swabs are to be disposed. In this embodiment, the clip comprises a metallic material and is preferably sterilizable.

With reference to FIG. 12, there is also provided an image recognition system for detecting and/or counting the number of swabs within a dock, the system comprising camera means, computer processing means, image recognition software with or without AI software. As shown in FIG. 12, the electronic means provides a further method of counting the number of swabs within a dock to assist the medical professional during a procedure. In one embodiment, the system may be connected to an electronic records system in respect of a patient. In the embodiment shown in FIG. 12, a clip is plugged into the dock and recorded on computer vision AI. The result is then displayed on a computer screen and can be seen by a medical professional. In this embodiment, the system shown has recognised that five swabs have been attached to the dock.

Preferably, the system is in communication with an app. Preferably, the system is programmed so as to know the correct number of swabs that should be present. It is preferred that the system does not allow data to be uploaded to the electronic records system until the correct number of swabs are present. Advantageously, the system allows for the independent verification of the number of swabs that are present.

The clip and/or dock comprise a visual identifier to enable them to be distinguished from a swab by the camera. Advantageously, the provision of a visual identifier on the clip and/or dock assists the user in identifying a swab after use (after use of a swab it can be difficult for a medical professional to see the swab since it is typically soiled with blood from a patient). Typically, the clip and/or dock are coloured in a manner such that they are distinguished from the swab and can be seen by the camera. In one embodiment, the clip and/or swab comprise a material having a bright colour that provides a contrast with the background to assist the camera in detecting the clip and/or swab.

In one embodiment, the system comprises a means for identifying if there is blood on the clip and/or swab so that the blood can be removed from the clip and/or swab. Preferably, the system only allows data to be uploaded when the correct number of swabs are present. This advantageously provides an independent means of checking that all of the swabs are present.

In one embodiment, the clip may comprise machine-readable information. In one embodiment the machine-readable information may be a barcode or a QR code which is printed on the clip or dock. Advantageously, the system provides a digital counter to determine the number of swabs that are attached to the dock. Preferably, the swabs are provided in a recess of the dock and once the correct number of swabs have been added the one or more clips are closed. Preferably, the closed clip comprises machine-readable information that may be read by the system.

The electronic means may be connected to an app which may be adapted to count cumulatively, detecting the clips using a camera and image recognition software with or without AI to detect and display the number of clips to a user.

FIG. 13a shows a top view of a swab counter system according to an embodiment of the invention. FIG. 13a shows a dock 450 comprising a plurality of recesses 460. A swab is placed into a recess within the dock and a clip 400 is provided to hold the swab 404 in place. The clip comprises a barcode 480 which provides a unique identifier. Once the swabs are placed into the recess the clips are then closed and the system reads the barcode. FIG. 13b shows a side view of the system of FIG. 13a.

FIG. 14 shows a side view of a system according to an embodiment of the invention wherein a clip 400 is provided that comprises a plurality of grooves 500. A swab 404 is placed into each groove and once each of the swabs has been placed into a corresponding groove the clip is closed. The clip comprises a barcode 480 which is then scanned to show that all of the swabs are present. This system advantageously provides a swab counter which independently verifies that all of the required swabs have been accounted for.

In use, swabs are placed into the recesses in the dock of FIG. 13 or the grooves in the clip of FIG. 14. Once the required number of swabs are held in place, the clip or clips are closed and the system reads the barcode to provide an independent means of verifying that all required swabs are present.

1-34. (canceled)

35. An apparatus for use in a surgical procedure, the apparatus comprising:
a dock for releasably securing a surgical device by means of a releasable tether of the device,
wherein the dock includes one or more ports, the one or ports each being configured such that when a tether is secured, a visible indication of attachment or detachment is provided by the port or the tether.

36. The apparatus according to claim 35, wherein a tactile indication is provided, as well as a visible indication.

37. The apparatus according to claim 35, further comprising one or more clip that are each configured for attachment to the proximal, in use, end of a tether of a surgical device, the or each clip being adapted to releasably connect the tether to the dock.

38. The apparatus according to claim 35, wherein the dock consists solely of a plurality of clips, each clip being adapted to connect with each other clip such that when all clips are connected, a complete dock is provided, the visible indication of the absence of a clip being provided by a gap between clips.

39. The apparatus according to claim 35, wherein the one or more clips are each removably attachable to a tether.

40. The apparatus according to claim 35, wherein the one or more clips are configured such that in use, the secured tethers will be disposed so that they are substantially parallel with one another.

41. The apparatus according to claim 41, wherein ports are arranged along a single side or face of the apparatus.

42. The apparatus according to claim 41, wherein the apparatus is configured to be used with substantially planar tethers, such as for example are present on surgical swabs, each tether is connectable to the apparatus in the same plane.

43. The apparatus according to claim 35, wherein the dock comprises a single body part, the part including a plurality of integrally molded ports, optionally or preferably, each port is configured such that a clip is receivable therein and retained therein through the resilience of molded material of the ports, or of the clips.

44. The apparatus according to claim 35, wherein each port may include a user visible aperture disposed to receive a correspondingly shaped formation of a clip, the aperture being filled by a part of the clip when the clip is correctly inserted in the port, optionally or preferably wherein the parts are adapted such that release of a clip is effected by a user action applied to the part of the clip in the aperture.

45. The apparatus according to claim 35, wherein each port comprises resilient arms describing a part circle and the corresponding clips comprise cylinders configured to fit within the arms, optionally or preferably wherein each cylinder includes a circumferential groove disposed to receive the arms therein.

46. The apparatus according to claim 35, wherein each port comprises a clip receiving aperture and a clip release aperture, and each corresponding clip comprises a release part adapted to engage with the release aperture and a tether securing location.

47. The apparatus according to claim 35, wherein each port includes a keeper adapted to be moved from a first unlocked condition to a second, locked condition, the arrangement being such that the keeper can only assume the locked condition when a clip is in situ in the port, optionally or preferably wherein the keeper and/or port is configured such that the position of the keeper, i.e. locked or unlocked, is visible to a user, and optionally or preferably, wherein the or each keeper is a door, openable about a hinge, the or each door including an aperture through which an associated clip may be viewed.

48. The apparatus according to claim 35, wherein:
a) the dock comprises a tray comprising one or more recesses, wherein the one or more recesses are shaped to accommodate a clip; or
b) the dock comprises card and wherein the one or more clips are reversibly attached to the card, or the dock comprises a clip comprising a plurality of recesses, wherein each recess is adapted to store one or more swabs. optionally or preferably the clip comprises an upper and a lower arm, wherein the upper and lower arm are joined by a hinged region, optionally or preferably the clip comprises a button which is adapted to release the one or more swabs from the one or more recesses.

49. The apparatus according to claim 35, wherein the apparatus is reusable.

50. A kit for use in surgery, the kit comprising the apparatus according to claim 35, and a plurality of surgical devices, each surgical device including a tether receivable in a clip.

51. A system comprising:
the apparatus according to claim 35;
a dock; and
a card.

52. A surgical method, comprising tethering surgical devices when in use in a procedure, to the apparatus according to claim 35.

53. An image recognition system for detecting and/or counting a number of swabs within a dock in accordance with claim 35, the system comprising:
camera means;
computer processing means, with or without AI software

54. The image recognition system according to claim 53, optionally or preferably further comprising a clip, and/or optionally or preferably one or more of the following:
a) wherein the system may be connected to an electronic records system in respect of a patient;
b) wherein the system is in communication with an app; and
c) wherein the clip and/or dock comprise a visual identifier, optionally or preferably wherein the visual identifier is a color that allow the clip and/or dock to be distinguished from a swab and/or wherein the clip and/or swab comprise machine-readable information.
